(19) United States
(12) Patent Application Publication
(10) Pub. No.: US 2018/0348193 A1
(43) Pub. Date: Dec. 6, 2018

(54) Test Sensor Systems And Methods For Using The Same

(71) Applicant: Ascensia Diabetes Care Holdings AG

(72) Inventors: Daniel V. Brown, Edwardsburg, MI (US); Matthew Odom, Mishawaka, IN (US); Jeffery S. Reynolds, New Fairfield, CT (US)

(21) Appl. No.: 15/504,272

(22) PCT Filed: Aug. 17, 2015
(86) PCT No.: PCT/US15/45520

§ 371 Date: Feb. 15, 2017

Related U.S. Application Data
(60) Provisional application No. 62/039,182, filed on Aug. 19, 2014.

(51) Int. Cl. G01N 33/487; G01N 27/327

(52) U.S. Cl. CPC G01N 33/48771; G01N 27/3272

(57) ABSTRACT

Systems and methods employ a test sensor that includes an identification feature, such as a resistive element or other detectable circuit element, which allows a measurement device to interrogate the test sensor and to determine whether the correct test sensor type is being used to collect a fluid sample. For example, some embodiments employ test sensors that a measurement device can identify according to a response received from the test sensors when the measurement device applies an electrical signal to the test sensors. By facilitating identification of a test sensor, embodiments help ensure that the desired test type, calibration codes, and/or assay sequence are applied to the fluid sample collected by the test sensor.

### CROSS-REFERENCE TO RELATED APPLICATION

This application claims priority to and the benefit of U.S. Provisional Patent Application No. 62/039,182, filed on Aug. 19, 2014, which is hereby incorporated by reference herein in its entirety.

### FIELD OF THE INVENTION

The present invention relates generally to systems and methods for determining one or more characteristics of a fluid sample. More specifically, the present invention relates to systems and methods that employ identifiable test sensors that ensure that the fluid sample is being collected with the correct test sensor.

### BACKGROUND OF THE INVENTION

The quantitative determination of analytes in body fluids is of great importance in the diagnoses and maintenance of certain physiological conditions. For example, persons with diabetes (PWDs) frequently check the glucose level in their bodily fluids. The results of such tests can be used to regulate the glucose intake in their diets and/or to determine whether insulin or other medication needs to be administered. A PWD typically uses a measurement device (e.g., a blood glucose meter) that calculates the glucose concentration in a fluid sample from the PWD, where the fluid sample is collected on a test sensor that is received by the measurement device.

### SUMMARY

Different types of measurement devices often use test sensors that appear to have the same or similar geometries and features. As such, there is a risk that the wrong type of test sensor will be used with a particular measurement device. According to aspects of the disclosure, systems and methods employ a test sensor, such as a resistive element or other detectable circuit element, which includes an identification feature that allows a measurement device to interrogate the test sensor and to determine whether the correct test sensor type is being used to collect a fluid sample. For example, some embodiments employ test sensors that a measurement device can identify according to a response received from the test sensors when the measurement device applies an electrical signal to the test sensors. By facilitating identification of a test sensor, embodiments help ensure that the desired test type, calibration codes, and/or assay sequence are applied to the fluid sample collected by the test sensor.

Still other aspects, features, and advantages of the present invention are readily apparent from the following detailed description, by illustrating a number of exemplary embodiments and implementations, including the best mode contemplated for carrying out the present invention. The present invention is also capable of other and different embodiments, and its several details can be modified in various respects, all without departing from the spirit and scope of the present invention. Accordingly, the drawings and descriptions are to be regarded as illustrative in nature, and not as restrictive. The invention is to cover all modifications, equivalents, and alternatives falling within the spirit and scope of the invention.

### BRIEF DESCRIPTION OF THE DRAWINGS

FIG. 1 illustrates an example system employing a reconfigurable measurement device.

FIG. 2A is a top view illustrating an example of an electrochemical test sensor having a detectable circuit element having a predetermined value to identify the test sensor.

FIG. 2B is a circuit diagram of the electrochemical test sensor in FIG. 2A;

FIG. 2C is a cross section front view illustrating an example of the electrochemical test sensor in FIG. 2A.

FIG. 3 illustrates an example resistor prepared by hand deposition of conducting polymer solution.

FIG. 4 is a table showing different parameters for the example resistors in FIG. 3.

FIG. 5 is a graph that illustrates the performance of dried polymer resistors.

The accompanying drawings, which are incorporated into this specification, illustrate one or more exemplary implementations of the inventions and, together with the detailed description, serve to explain the principles and applications of these inventions. The drawings and detailed description are illustrative, not limiting, and can be adapted without departing from the spirit and scope of the inventions.

### DETAILED DESCRIPTION

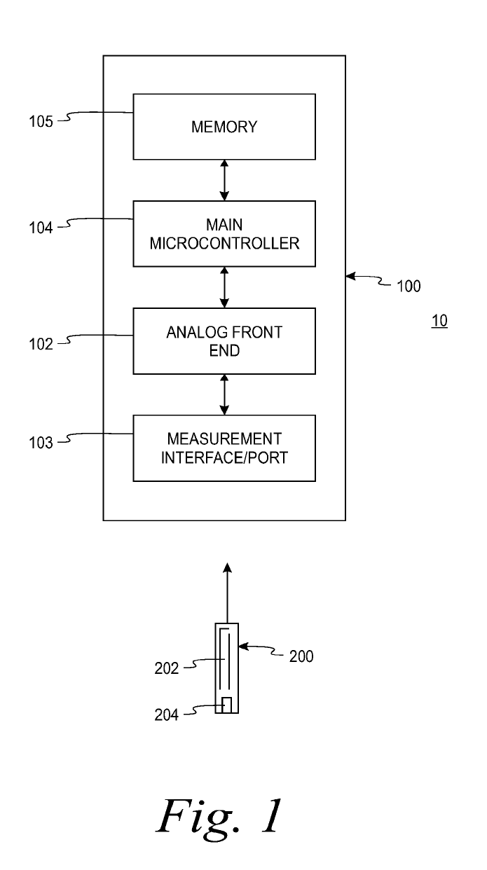

Referring to FIG. 1, an example system 10 employing a measurement device 100 and a test sensor 200 is illustrated. In particular, the measurement device 100 includes an analog front end 102, a measurement interface 103, a main microcontroller 104, and a memory 105. The analog front end 102 is coupled to the measurement interface 103, which includes a port or opening and hardware to receive and engage the test sensor 200. The test sensor 200 collects a fluid sample for analysis by the measurement device 100. In some embodiments, for example, the measurement device 100 measures the concentration of an analyte in the fluid sample. The fluid sample may include, for example, a whole blood sample, a blood serum sample, a blood plasma sample, other body fluids like ISF (interstitial fluid), saliva, and urine, as well as non-body fluids. Analytes that may be analyzed include glucose, lipid profiles (e.g., cholesterol, triglycerides, LDL and HDL), microalbumin, hemoglobin Al_{c}, fructose, lactate, or bilirubin. In general, aspects of the present invention may be employed to measure one or more characteristics of a sample, such as analyte concentration, enzyme and electrolyte activity, antibody titer, etc.

For example, a user may employ a lancing device to pierce a finger or other area of the body to produce a blood sample at the skin surface. The user may then collect this blood sample by placing the test sensor into contact with the sample. The test sensor contains a reagent which reacts with the sample to indicate the concentration of an analyte in the sample. In engagement with the test sensor 200, the measurement interface 103 allows the reaction to be measured by the analog front end 102.

As shown in FIG. 1, the test sensor 200 may be an electrochemical test sensor. An electrochemical test sensor includes a plurality of electrodes 202 and a fluid-receiving area 204 that receives the fluid sample and includes appropriate reagent(s) (e.g., enzyme(s)) for converting an analyte of interest (e.g., glucose) in a fluid sample (e.g., blood) into a chemical species that produces an electrical current which is electrochemically measurable by the components of the electrode pattern. In such cases, the measurement interface 103 allows the analog front end 102 to be coupled to the electrodes 202 of the test sensor 200, and the analog front end 102 receives the electrical current from the measurement interface 103. The electrodes 202 are arranged in an appropriate circuit to deliver the electrical current to the analog front end 102. For example, the electrodes 202 may include a working electrode and counter electrode, where the working electrode measures the electrical current when a potential is applied across the working and counter electrodes. Other types of electrodes or electrical leads (e.g., a calibration lead or a hematocrit electrode lead) may also be employed on the test sensor 200.

In general, the analog front end 102 is employed to measure characteristic(s) of fluid samples received via the measurement interface 103. Also coupled to the analog front end 102, the main microcontroller 104 controls operative aspects of the measurement device 100. For example, the main microcontroller 104 can manage the measurement sequence that determines how the actual electrochemical measurement is performed and how the electrical current is obtained by the analog front end 102 from the respective measurement interface 103. In addition, the main microcontroller 104 can determine how the raw signal received by the analog front end 102 is converted with a calculation sequence into a final measurement value (e.g., blood glucose concentration expressed as milligrams per deciliter (mg/dL)) that can be communicated to the user, e.g., by a display. Although the analog front end 102 and the main microcontroller 104 are shown separately in FIG. 1, it is contemplated that the main microcontroller 104 in alternative embodiments may include a sufficient analog front end to measure characteristic(s) of a fluid sample received via the at least one measurement interface 103. In addition, it is contemplated that the main controller 104 shown in FIG. 1 may generally represent any number and configuration of processing hardware and associated components required to manage the operation of the measurement device 100.

The memory 105 (e.g., non-volatile memory) may include any number of storage devices, e.g., EEPROM, flash memory, etc. The memory 105 may store measurement data. In addition, the memory 105 may store data, e.g., firmware, software, algorithm data, program parameters, calibration data, lookup tables, etc., that are employed in the operation of other components of the measurement device 200. In this example, the memory 105 may store a lookup table of predetermined values that are associated with detectable circuit elements for desired/acceptable test sensors as a form of identification of the test sensors.

Different types of measurement devices often use test sensors that appear to have the same or similar geometries and features. As such, there is a risk that the wrong type of test sensor will be used with a particular measurement device. According to aspects of the present invention, systems and methods employ a test sensor that includes an identification feature that allows a measurement device to interrogate the test sensor and to determine whether the correct test sensor type is being used to collect a fluid sample. For example, some embodiments employ test sensors that a measurement device can identify according to a response received from the test sensors when the measurement device applies an electrical signal to the test sensors. By facilitating identification of a test sensor, embodiments help ensure that the desired test type, calibration codes, and/or assay sequence are applied to the fluid sample collected by the test sensor.

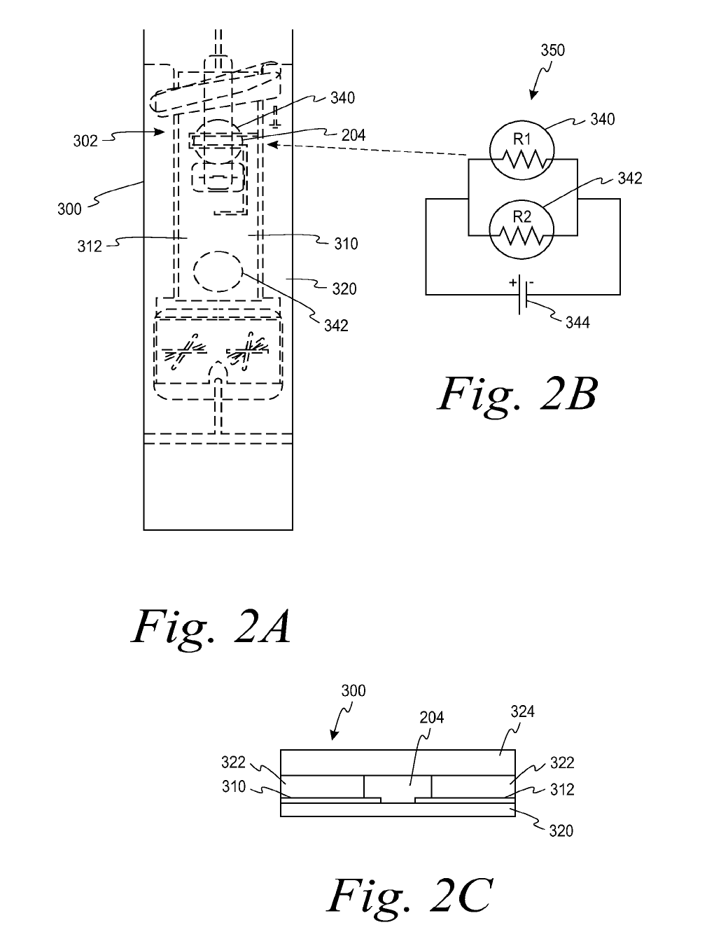

Referring to FIG. 2A, a top view of an example electrochemical test sensor 300 is illustrated. The electrochemical sensor 300 is illustrated functionally by a circuit diagram 350 in FIG. 2B where resistor 340 (R₁) is the resistance of the circuit path that includes the electrodes 302 (e.g., working and counter electrodes 310 and 312, respectively). The fluid-receiving area 204 holds a fluid sample and the electrodes 310 and 312 are in contact with the fluid sample. The electrodes 302 are formed on a base 320. In this example, a spacer 322 is located between a lid 324 and the base 320 as shown in the front cross section view in FIG. 2C.

In a dry test sensor 300 (before application of the fluid sample), this resistance approaches infinity. When a fluid sample is applied to the fluid receiving area 204 of the test sensor 300, the resistance between the electrodes 310 and 312 immediately drops. Applying Ohm's Law, the circuit resistance is on the order of 2.5 megohms when a 250 mV potential 344 is applied to the electrodes 302 and a 100 nA current is generated in the presence of a low concentration of glucose. If a second resistor 342 (R₂) is placed in parallel to resistor R₁ 340 as shown in FIG. 2A and 2B, the circuit resistance is approximated as R₁R₂/(R₁+R₂). In the initial dry state, a potential applied to the sensor will cause current to flow in resistor R₂ 342. After the fluid sample is applied to the sensor 300 and resistor R₁ 340 drops correspondingly to a low value, most of the current flows through the resistor R₁ 340. By manipulating the resistance of resistor R₂ 342 appropriately, a circuit of the measurement device may be configured to interrogate the dry test sensor 300 prior to testing and identify the test sensor 300 based on the resistance of resistor R₂ 342. If an appropriate resistance is not detected (i.e., high but not an open circuit), the measurement device can reject the sensor.

In general, it is contemplated that any resistive element or detectable circuit element may be employed on a test sensor, so that a measurement device can identify the test sensor. In particular, R₂ need not be implemented parallel to R₁ of the electrodes 302 as shown in FIG. 1. A resistive element may be implemented across a calibration lead or a hematocrit electrode lead of a test sensor. Alternatively, a resistive element may be implemented as a solution of conducting polymer that is deposited across two conductor leads and dried, much the same as the deposition and drying process used to process the enzyme chemistry. Or instead, a resistive element may be implemented by striping a resistive solution on the underside of spacer tape of a test sensor, and then laminating the spacer tape onto the patterned electrode.

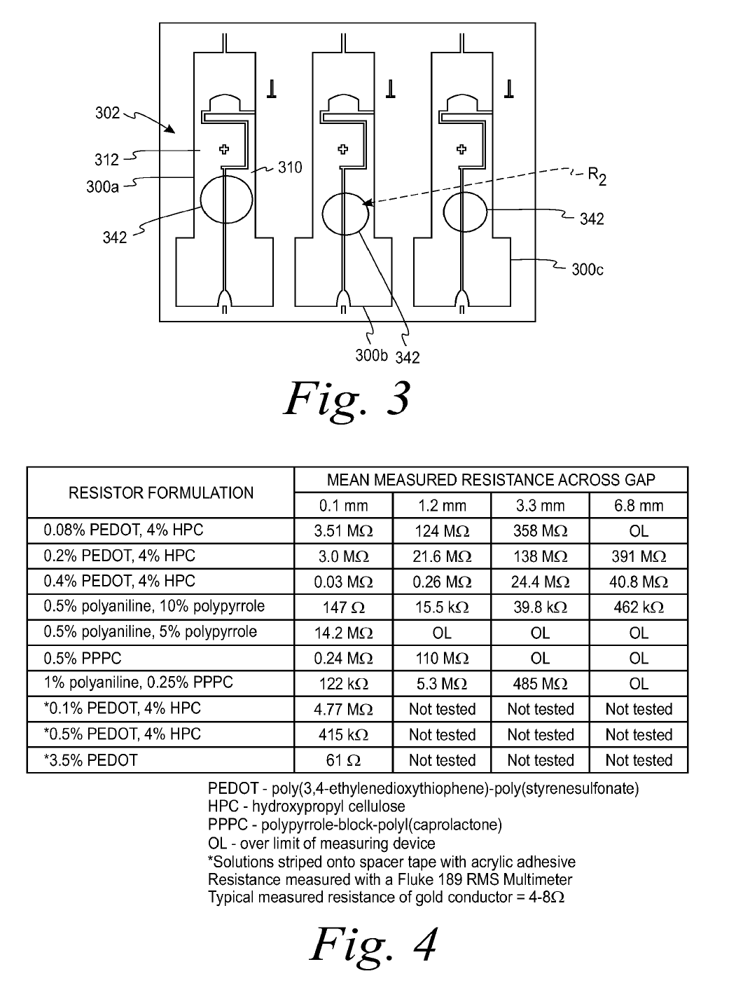

FIG. 3 illustrates several test sensors 300a, 300b and 300c laid out in sequence on a web. Each of the test sensors 300a, 300b and 300c includes a resistor 342 such as resistor R₂ in FIG. 2A. In particular, resistors R₂ 342 on the test sensors 300a, 300b and 300c were prepared by hand deposition of conducting polymer solution across a 0.1 mm gap between the counter electrode 310 and the working electrode 312. The conductive patterns are sputtered gold that has been laser-ablated to achieve a pattern of electrodes 310 and 312 and conductors. A solution of 0.4% poly (3,4-ethylenedioxythiophene)-poly(styrenesulfonate) (PEDOT) [Aldrich] and 4.0% hydroxypropyl cellulose (HPPC) [Ashland] in water may be hand-deposited onto the test sensor 300 across the conductor leads 302. After drying, the mean resistance across the 0.1 mm gap, for example, may be approximately 30 kΩ. By reducing the amount of conducting polymer from 0.4 to 0.08%, the resistance may increase to approximately 3.51 MΩ. Increasing the gap from approximately 0.1 to approximately 6.8 mm with the latter solution resulted in a mean resistance of 358 MΩ. Several other mixtures of conducting polymers are also contemplated, including the implementation of striping the conducting polymer on spacer adhesive.

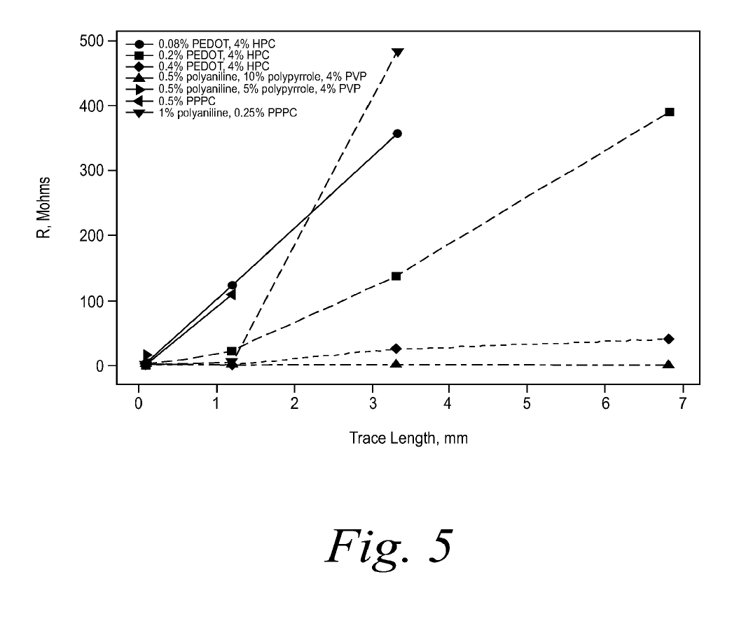

FIG. 4 is a table showing the results of testing on a series of mixtures of conducting polymers of dried polymer resistors such as the resistors 300a, 300b and 300c in FIG. 3. The table in FIG. 4 shows the mean measured resistance across different gaps based on different resistor formulations using different combinations of PEDOT, HPC, polypyrrole-block-polyl (caprolactone) (PPPC) and polyaniline. FIG. 5 is a graph showing the resistance performance of the dried polymer resistors having different combination of materials in FIG. 4. The data in FIGS. 4 and 5 clearly indicate that resistance of a deposited or striped solution is “tunable” over a wide range, depending on conducting polymer, concentration, and circuit geometry. It is envisioned that many other polymers other than those named here may be employed. It also is contemplated that different levels of measured resistance could indicate different types of sensors.

In summary, aspects of the present invention address the potential problem of users attempting to use an incorrect test sensor for a particular test/measurement device. In response to this problem, example embodiments provide a resistive element across two test sensor electrodes/leads so that a measurement device can measure a finite resistance that identifies the test sensor during a test initialization sequence. The options for forming this resistive element on a test sensor include:

Depositing a liquid reagent-style solution and drying (in a process similar to enzyme reagent deposition).

Striping a resistive conductor across the spacer tape that is assembled to the base of the test sensor.

Forming a serpentine conductor path in the base of the test sensor.

Screen printing or other conductive ink printing.

In this example, the controller 104 in FIG. 1 may access the memory 105 for predetermined values that are associated with detectable circuit elements when a test sensor such as the test sensor 200 is interfaced with the measurement device 100. The controller 104 detects a predetermined value from the detectable circuit element of the test sensor 200 and compares that detected predetermined value with the table of stored values that are associated with acceptable (“correct”) test sensors. If there is a match, the controller 104 initiates the measurement sequence to the test sensor 200 for measuring a fluid sample in the test sensor 200. If the predetermined value detected by the controller 104 does not match any of the predetermined values associated with the identification of a correct test sensor, the controller 104 will prevent the measurement sequence.

The advantages of aspects of the present invention therefore include:

Implementation is on a test sensor, not a measurement device, so that it can be modified independently of the installed measurement device base.

Implementation can be made by positioning the resistor on a base or on a spacer of a test sensor.

The resistive element may be employed at various locations on the test sensor depending on the particular test sensor design.

The resistance of the element can be configured to match the test sensor chemistry.

While the invention is susceptible to various modifications and alternative forms, specific embodiments and methods thereof have been shown by way of example in the drawings and are described in detail herein. It should be understood, however, that it is not intended to limit the invention to the particular forms or methods disclosed, but, to the contrary, the intention is to cover all modifications, equivalents and alternatives falling within the spirit and scope of the invention.

1. An electrochemical test sensor to collect a fluid sample for analysis, the test sensor comprising:
a detectable circuit element having a predetermined value to identify the test sensor;
a plurality of electrodes operable to interface with a measurement device configured to interrogate the test sensor by applying a signal to the detectable circuit element to determine the identity of the test sensor based on the predetermined value; and
a fluid receiving area for receiving the fluid sample, wherein the plurality of electrodes contacts the fluid sample upon receipt of the fluid sample in the fluid receiving area.

2. The test sensor of claim 1, wherein the detectable circuit element is a resistive element.

3. The test sensor of claim 2, wherein the resistive element is coupled to one of the plurality of electrodes.

4. The test sensor of claim 1, wherein the plurality of electrodes includes at least one of a working electrode and a counter electrode.

5. The test sensor of claim 2, wherein the resistive element is composed of a conducting polymer solution.

6. The test sensor of claim 5, wherein the predetermined value is a function of at least one of the amount of conducting polymer solution, the concentration of the conducting polymer solution or the geometry of the resistive element.

7. The test sensor of claim 1, wherein the detectable circuit element is wired in parallel with the plurality of electrodes.

8. The test sensor of claim 1, further comprising:
a base, the plurality of electrodes being formed on the base;
a lid; and
a spacer located between the base and the lid, wherein the detectable circuit element is positioned on the spacer.

9. The test sensor of claim 5, wherein the conducting polymer solution includes at least one of the group of poly (3,4-ethylenedioxythiophene)-poly(styrenesulfonate) (PEDOT), hydroxypropyl cellulose (HPC), polypyrrole-block-polyl (caprolactone) (PPPC) and polyaniline.

10. A measurement device for measuring a fluid sample in a test sensor, the test sensor having a detectable circuit element having a predetermined value associated with the identification of the test sensor, the measurement device comprising:
a measurement interface for interfacing with the test sensor;
an analog interface for applying a signal to the detectable circuit element; and
a controller coupled to the analog interface, the controller controlling a measurement sequence to the test sensor,
wherein the controller applies an electrical signal to the detectable circuit element via the analog interface and detects the predetermined value from the detectable circuit element to identify the test sensor.

11. The measurement device of claim 10, further comprising a memory coupled to the controller.

12. The measurement device of claim 11, wherein the memory stores measurement data and operational data for the measurement device operating with the identified test sensor.

13. The measurement device of claim 10, wherein the controller prevents the measurement sequence if the predetermined value detected by the controller does not match the identification of the correct type of test sensor.

14. The measurement device of claim 10, wherein the measurement sequence measures the concentration of an analyte in the fluid sample via an input signal to the test sensor and receives an electrical current via the analog interface.

15. A fluid sample analysis system comprising:
a test sensor to collect a fluid sample for analysis, the test sensor including:
a plurality of electrodes; and
a detectable circuit element having a predetermined value to identify the test sensor; and
a measurement device having a measurement interface contacting the plurality of electrodes of the test sensor, the measurement device interrogating the test sensor by applying a signal to the detectable circuit element to determine the identity of the test sensor based on the predetermined value.

16. A method of determining the identification of a test sensor, the test sensor including a detectable circuit element, the method comprising:
interfacing a test sensor to a measurement device;
applying an electrical signal to the detectable circuit element;
determining an output value from the detectable circuit element associated with the identification of the test sensor; and
determining whether the test sensor is a correct type of test sensor based on the output value.
